(19) United States
(12) Patent Application Publication
(10) Pub. No.: US 2020/0214969 A1
(43) Pub. Date: Jul. 9, 2020

(54) Compositions and Methods for Topical Skin Treatment

(71) Applicant: Asher Shuan, Giva'taim (IL)

(72) Inventor: Asher Shuan, Giva'taim (IL)

(21) Appl. No.: 16/239,557

(22) Filed: Jan. 4, 2019

(51) Int. Cl. A61K 8/9789; A61K 8/9794; A61K 8/34; A61K 8/36; A61K 8/49; A61K 8/37; A61K 8/67; A61Q 19/08; A61K 8/02

(52) U.S. Cl. CPC A61K 8/9789; A61K 8/9794; A61K 8/34; A61K 8/345; A61K 8/36; A61K 8/49; A61K 2800/43; A61K 8/347; A61K 8/678; A61K 8/676; A61Q 19/08; A61K 8/0204; A61K 8/37

(57) ABSTRACT

Disclosed is a biocompatible composition—for topical skin treatment of skin wrinkling, cellulite and/or for breast firming. The topical composition comprises a combination of active materials/substances including, or derived from: Pink Pepperslim (Schinus terebinthifolius), Black Cohosh (Actaea racemosa) and/or Nettle (Urtica dioica).

### FIELD OF THE INVENTION

The present invention generally relates to the fields of topical medicines and topical skin treatment. More specifically, the present invention relates to compositions and methods for topical skin treatment.

### BACKGROUND

The present invention relates to cosmetic compositions for improving the appearance of skin and to methods of application and preparation thereof.

A wrinkle, also known as a rhytide, is a fold, ridge or crease in the skin or on fabric. Skin wrinkles typically appear as a result of aging processes such as glycation, habitual sleeping positions, loss of body mass, or temporarily, as the result of prolonged immersion in water. Age wrinkling in the skin is promoted by habitual facial expressions, aging, sun damage, smoking, poor hydration, and various other factors.

Wrinkled skin presents a perceived problem for many people who wish to improve their appearance. There is an enormous demand for cosmetic formulations that reduce wrinkles or the appearance of wrinkles.

Current evidence suggests that tretinoin decreases cohesiveness of follicular epithelial cells, although the exact mode of action is unknown. Additionally, tretinoin stimulates mitotic activity and increased turnover of follicular epithelial cells. Tretinoin is better known by the brand name Retin-A.

Topical glycosaminoglycans supplements can help to provide temporary restoration of enzyme balance to slow or prevent matrix breakdown and consequent onset of wrinkle formation. Glycosaminoglycans (GAGs) are produced by the body to maintain structural integrity in tissues and to maintain fluid balance. Hyaluronic acid is a type of GAG that promotes collagen synthesis, repair, and hydration. GAGs serve as a natural moisturizer and lubricant between epidermal cells to inhibit the production of matrix metalloproteinases (MMPs).

Dermal fillers are injectable products frequently used to correct wrinkles, and other depressions in the skin. They are often a kind of soft tissue designed to enable injection into the skin for purposes of improving the appearance. The most common products are based on hyaluronic acid and calcium hydroxylapatite.

Botulinum toxin is a neurotoxin protein produced by the bacterium Clostridium botulinum. Botox is a specific form of botulinum toxin manufactured by Allergan for both therapeutic and cosmetic use. Besides its cosmetic application, Botox is used in the treatment of other conditions including migraine headache and cervical dystonia (spasmodic torticollis—a neuromuscular disorder involving the head and neck).

Dysport, manufactured by Ipsen, received FDA approval and is now used to treat cervical dystonia as well as glabellar lines in adults. In 2010, another form of botulinum toxin, one free of complexing proteins, became available to Americans. Xeomin received FDA approval for medical indications in 2010 and cosmetic indications in 2011.

Botulinum toxin treats wrinkles by immobilizing the muscles which cause wrinkles. It is not appropriate for the treatment of all wrinkles; it is indicated for the treatment of glabellar lines (between the eyebrows) in adults. Any other usage is not approved by the FDA and is considered off-label use.

Laser resurfacing is FDA-cleared skin resurfacing procedure in which lasers are used to improve the condition of the skin. Two types of lasers are used to reduce the appearance of fine lines and wrinkles on the face; an ablative laser that removes thin layers of skin and a non-ablative laser that stimulates collagen production. Non-ablative lasers are less effective than ablative ones but they are less invasive and recovery time is short. After the procedure people experience temporary redness, itching and swelling.

There remains a need, in the fields of topical medicines, cosmetics and topical skin treatment for compositions and methods for topical skin treatment, for wrinkle prevention and for anti-aging.

### SUMMARY OF THE INVENTION

The present invention relates to topical compositions and methods for treating skin wrinkling, cellulite and/or for breast firming, through topical composition application.

According to some embodiments of the present invention, there may be provided a biocompatible composition comprising a combination of active materials/substances including, or derived from: Pink Pepperslim (Schinus terebinthifolius), Black Cohosh (Actaea racemosa) and/or Nettle (Urtica dioica).

The topical composition, in accordance with some embodiments, may comprise or consist of one or more additional substances/agents such as, but not limited to: a carrier, which may be a solvent; a chelating agent; a humectant; a thickener; an emollient; an emulsifier; a skin conditioning agent; an anti-inflammatory agent; an antioxidant; a pH adjuster; and/or a preservative. In addition, the topical composition may also comprise or consist of one or more biocompatible carrier, excipient, filler and/or diluent.

For example, the topical composition may optionally comprise or consist of a combination of the following additional substances/agents: Water, Alcohol-oil mixture, Dimethicone (Silicone based oil), Cetyl Alcohol (oil mixing substance), Shea butter, Glycerin (skin moisturizer or moisture retainer/holder), Acrylates-Fixative substance and/or Sodium Hydroxide (base substance).

The additional substances/agents included in the topical compositions may, for example: affect the texture (e.g. cream, soften, smoothen, stabilize) of the topical composition, extend the shelf life of the topical composition, preserve the characteristics and/or quality of the topical composition; and/or facilitate, or affect the rate of, absorption of the active materials of the topical composition, upon its topical application to the skin of a subject.

The present invention further relates to a method of treating, preventing or ameliorating skin wrinkling, cellulite, or a symptom thereof in a subject and/or for breast firming—comprising or consisting of topically administering to the subject a therapeutically effective amount of the a biocompatible topical composition described herein.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

Functions, operations, components and/or features described herein with reference to one or more embodiments, may be combined with, or may be utilized in combination with, one or more other functions, operations, components and/or features described herein with reference to one or more other embodiments, or vice versa.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “includes”, “including”, “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention, and is not intended to limit the invention to the specific embodiments illustrated by the figures or description below.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application. For example, “an” astringent component refers to both one astringent component or to a mixture comprising two or more astringent components.

Unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained.

Throughout the application, descriptions of various embodiments use “comprising” language; however, it will be understood by one of skill in the art, that in some specific instances, an embodiment can alternatively be described using the language “consisting essentially of” or “consisting of” Other terms as used herein are meant to be defined by their well-known meanings in the art.

Lastly, throughout the specification, discussions utilizing terms such as “cream”, “lotion”, “ointment”, “gel”, or the like, may refer to a topical composition for skin application—and may be used interchangeably. The components, compositions, solutions, and methods in the specification and accompanying tables are described in the context of a composition for topical skin application, this however, is not to limit in any way the teachings herein to topical and/or skin applied compositions—all or some of which teachings may be likewise applicable to other types or methods of treatment, application and/or remediation.

The present invention relates to topical compositions and methods for treating skin wrinkling, cellulite and/or for breast firming, through topical composition application.

According to some embodiments of the present invention, there may be provided a biocompatible composition comprising a combination of active materials/substances including, or derived from: Pink Pepperslim (Schinus terebinthifolius), Black Cohosh (Actaea racemosa) and/or Nettle (Urtica dioica).

The topical composition, in accordance with some embodiments, may comprise or consist of one or more additional substances/agents such as, but not limited to: a carrier, which may be a solvent; a chelating agent; a humectant; a thickener; an emollient; an emulsifier; a skin conditioning agent; an anti-inflammatory agent; an antioxidant; a pH adjuster; and/or a preservative. In addition, the topical composition may also comprise or consist of one or more biocompatible carrier, excipient, filler and/or diluent.

For example, the topical composition may optionally comprise or consist of a combination of the following additional substances/agents: Water, Alcohol-oil mixture, Dimethicone (Silicone based oil), Cetyl Alcohol (oil mixing substance), Shea butter, Glycerin (skin moisturizer or moisture retainer/holder), Acrylates-Fixative substance and/or Sodium Hydroxide (base substance).

The additional substances/agents included in the topical compositions may, for example: affect the texture (e.g. cream, soften, smoothen, stabilize) of the topical composition, extend the shelf life of the topical composition, preserve the characteristics and/or quality of the topical composition; and/or facilitate, or affect the rate of, absorption of the active materials of the topical composition, upon its topical application to the skin of a subject.

The present invention further relates to a method of treating, preventing or ameliorating skin wrinkling, cellulite, or a symptom thereof in a subject and/or for breast firming—comprising or consisting of topically administering to the subject a therapeutically effective amount of the a biocompatible topical composition described herein.

### Biocompatible Topical Compositions

The biocompatible topical compositions described herein, in accordance with some embodiments, may be produced/provided in various forms, including, but not limited to: a gel, a cream, a lotion, an ointment, a foam, an aerosol, a powder, a solution, an emulsion, and/or a serum.

### Active Substances

A topical composition, in accordance with embodiments of the present invention, may comprise any combination of the following substances. Some or all of these substances may penetrate the epidermis skin layer and thus be introduced into the blood through the capillaries/vessels of the dermis—and may thus be regarded as active substances. Some or all of the active substances described herein, in accordance with some embodiments, may be added to the topical composition in the form of an extract.

According to some embodiments, the relative amounts/weights of the active substances—within some or all of the topical composition formulas and/or topical composition substance/component amount/weight ranges provided herein—may relate to the relative amount/weight of actual/purely/only active substance within the final topical composition and may thus exclude other ingredients/parts of the active substance component. For example, an active material extract added to the topical composition may also include oil, water and/or other non-active components.

A topical composition, in accordance with some embodiments of the present invention, may comprise Pink Pepperslim (Schinus terebinthifolius). The Pink Pepperslim (Schinus terebinthifolius)—produced from a Brazilian forest fruit (Also known as ‘Pink Gold’)—may, according to some embodiments, be added to the topical composition in the form of an extract.

The Pink Pepperslim in the composition may increase blood flow in skin area(s) where applied (in some examples by up to 15 times). According to some embodiments, the concentration of the active material within the Pink Pepperslim extract; and/or the concentration of the extract within the topical composition—may be set to an amount(s) which minimizes, or completely prevents, the penetration of the active material in the extract through the hypodermis layer of the treated subject's skin and possibly affecting other physiological processes such as the subject's metabolism.

The Pink Pepperslim (Schinus terebinthifolius) may, for example, be present in the topical composition at a concentration between: 0% to about 4% by weight; 0% to about 2.5% by weight; 0% to about 0.1% by weight; 0% to about 0.2% by weight; about 0.09% to about 0.11% by weight; about 0.05% to about 0.15% by weight; and/or in any other amount within any of the above ranges.

According to some embodiments, the Pink Pepperslim (Schinus terebinthifolius) may be present in the topical composition—for example, as part of a topical composition intended for face application—at a concentration between: 0% to about 0.05% by weight; 0% to about 0.1% by weight; 0% to about 0.2% by weight; 0% to about 0.5% by weight; about 0.09% to about 0.11% by weight; about 0.07% to about 0.13% by weight; and/or in any other amount within any of the above ranges.

A topical composition, in accordance with some embodiments of the present invention, may comprise Black Cohosh (Actaea racemosa). The Black Cohosh (Actaea racemosa)—produced from an American plant—may, according to some embodiments, be added to the topical composition in the form of an extract.

The Black Cohosh in the composition may stabilize, or otherwise affect, hormonal activity and relief related symptoms (e.g. heat waves due to hormonal instability in women). According to some embodiments, Black Cohosh extract/component may be used as part of the topical composition. According to some embodiments, Black Cohosh (known as milder) extract/component and other Cohosh types and/or colors extracts/components may be used in any combination as part of the topical composition.

The Black Cohosh (Actaea racemosa) may be present in the topical composition at a concentration between: 0% to about 2% by weight; 0% to about 0.02% by weight; about 0.1% to about 2% by weight; about 0.01% to about 0.03% by weight; and/or in any other amount within any of the above ranges.

A topical composition, in accordance with some embodiments of the present invention, may comprise Nettle (Urtica dioica). The Nettle (Urtica dioica) may, according to some embodiments, be added to the topical composition in the form of an extract. The Nettle in the composition may, in accordance with some embodiments, increase blood flow in skin area(s) where applied.

The Nettle (Urtica dioica) may be present in the topical composition at a concentration between: 0% to about 2% by weight; 0% to about 0.02% by weight; about 0.1% to about 2% by weight; about 0.01% to about 0.03% by weight; and/or in any other amount within any of the above ranges.

A topical composition, in accordance with some embodiments of the present invention, may comprise Shea Butter (Butyrospermum parkii).

The Shea Butter (Butyrospermum parkii) may, according to some embodiments, preserve skin moisture by creating a ‘seal’ preventing the escape of water from the skin. In addition, as the Shea Butter does not react with alkalis it tends to preserve its skin moisturizing properties. The moisturizers in the Shea Butter share properties with those extracted by the skin's Sebaceous glands and it is thus considered a Natural Moisturizing Factor (NMF), which helps preserve the inter cell structure of the epidermis, keeping/stopping germs, facilitating skin healing abilities and preventing skin rash/irritation.

Shea Butter may improve the look of dry skin, protect the skin from cold weather conditions, act as an anti-inflammatory agent, act as an anti-oxidant, and/or relief skin itching, irritating and burning. Shea Butter also protects the skin by absorbing UVB rays.

The Shea Butter may be present in the topical composition at a concentration between: 0% to about 2.5% by weight; about 0.1% to about 2.5% by weight; about 2% to about 3% by weight; about 2.4% to about 2.6% by weight; and/or in any other amount within any of the above ranges.

A topical composition, in accordance with some embodiments of the present invention, may comprise Persea gratissima. The Persea gratissima may for example be produced from pressured/pressed dried Avocado and may act as a carrier agent to the skin. The Persea gratissima is reach in acids/fatty-acids and may act as an emulsifier.

The Persea gratissima may be present in the topical composition at a concentration between: 0% to about 2.5% by weight; about 0.1% to about 2.5% by weight; about 2% to about 3% by weight; about 2.4% to about 2.6% by weight; about 1% to about 4% by weight and/or in any other amount within any of the above ranges.

A topical composition, in accordance with some embodiments of the present invention, may comprise Hamamelis (Witch-hazel). The Hamamelis may act as a skin irritation reliever.

The Hamamelis (Witch-hazel) may be present in the topical composition at a concentration between: 0% to about 2% by weight; about 0.1% to about 2% by weight; about 1.5% to about 2.5% by weight; about 1.9% to about 2.1% by weight; about 1% to about 3% by weight and/or in any other amount within any of the above ranges.

A topical composition, in accordance with some embodiments of the present invention, may comprise Aloe Vera. The Aloe Vera may act as a skin irritation reliever.

The Aloe Vera may be present in the topical composition at a concentration between: 0% to about 2% by weight; about 0.1% to about 2% by weight; about 1.5% to about 2.5% by weight; about 1.9% to about 2.1% by weight; about 1% to about 3% by weight and/or in any other amount within any of the above ranges.

A topical composition, in accordance with some embodiments of the present invention, may comprise Wild Yam (Dioscorea villosa). The Wild Yam includes Phytoestrogens—the plant hormones—which have been shown to interact with hormone receptors in human bodies, while having estrogens and/or phytoestrogens effects on the skin.

The Wild Yam (Dioscorea villosa) may be present in the topical composition at a concentration between: 0% to about 1% by weight; about 0.1% to about 1% by weight; about 0.5% to about 1.5% by weight; about 0.9% to about 1.1% by weight; and/or in any other amount within any of the above ranges.

A topical composition, in accordance with some embodiments of the present invention, may comprise Oat (Avena sativa). The Oat, or extracts thereof, may soothe skin conditions and are known to have emollient properties. It has also shown to have balancing effects on menstruation among women.

The Oat (Avena sativa) may be present in the topical composition at a concentration between: 0% to about 2% by weight; about 0.1% to about 2% by weight; about 1.5% to about 2.5% by weight; about 1.9% to about 2.1% by weight; about 1% to about 3% by weight and/or in any other amount within any of the above ranges.

### Additional Substances

A topical composition, in accordance with embodiments of the present invention, may further comprise any combination of the following additional substances. One or more of the additional substances may, for example: act-as/constitute the base of the topical composition; create/improve the texture of the topical composition (e.g. make creamy, softy, smooth); extend the life of the composition while preserving its properties/qualities; and/or add/change specific properties thereof, wherein changed properties, in accordance with some embodiments, may affect the absorption/activity characteristics (e.g. increase absorption rate into the blood) of one or more of the composition's active substances described herein.

The additional substances may be selected from, but are not limited to, the following material/substance types: carriers, solvents, thickeners, conditioning agents, chelating agents, skin conditioning agents, emulsifiers, emollients, exfoliating agents, neurogenic agents, inflammatory management agents, vascular repair agents, antioxidants, sebum suspension agents, preservatives and/or pH adjusters.

A topical composition, in accordance with embodiments of the present invention, may, for example, comprise any combination of the following substances, in addition to a combination of active materials described herein: Alcohol—Isopropyl, Propanol, Propylene Glycol and/or other—for facilitating the mixing of fatty substances in the composition and to generate humidity; Acids—Acetic acid, Alpha acid and/or other; Preservatives—Organic acid, Phenoxyethanol, Sothiazolinones, Formaldehyde, Parabens, Ethylhexylglycerin, BHT (Butylated Hydroxytoluene) and/or other; and/or Vitamins—Vitamin E, Vitamin C and/or other.

A topical composition, in accordance with some embodiments of the present invention, may comprise deionized water and/or pasteurized water. The relative amount/weight of water in an exemplary topical composition, in accordance with some embodiments, may be 65%, 68%, 70% and/or any value roughly around or in-between these values. A matrix may be utilized for raising water amount/weight—for example, from 68% to 70%.

A topical composition, in accordance with some embodiments, may be applied/administered through/using a mix droplet, for example, a 2-5 mix droplet.

A topical composition, in accordance with some embodiments, may comprise a brown, caramel, mocha, bez and/or other color pigment—for giving the topical composition a skin like color or shade.

A topical composition, in accordance with some embodiments, may comprise an insect repellent component. According to some embodiments, the relative amount of insect repellent within an exemplary topical composition may be set reverse-proportionally to the amount of Black Cohosh (Actaea racemosa) it includes. The relative repellent amount may accordingly be decreased, or completely avoided, as the amount of Black Cohosh (Actaea racemosa) in the composition is increased.

A topical composition, in accordance with some embodiments, may comprise a perfume fixative, for example Phenoxyethanol.

A topical composition, in accordance with some embodiments, may comprise a preservative, for example Ethylhexylglycerin.

A topical composition, in accordance with some embodiments, may comprise an antioxidant, for example BHT (Butylated Hydroxytoluene).

A topical composition, in accordance with embodiments of the present invention, may further comprise a combination of smells/scents/fragrances. Smells/scents/fragrances may, for example, include any combination of the following exemplary fragrances/substances/extracts: Butylphenyl, Methylpropional, Coumarin, Benzyl Salicylate, Limonene, Linalool, Geraniol, Amyl Cinamal, Citronellol, Hexy Cinamal and/or others.

### Methods of Treatment

A topical composition, in accordance with embodiments of the present invention, may for example be applied to the skin of a subject by spreading a layer (e.g. very thin layer) of the composition (e.g. in a cream form). Active materials in the composition may then penetrate through the porosities and be-introduced-into/stimulate/interact-with the blood, while the oily materials in the composition at least partially block the porosities, preventing/minimizing the loss of humidity out of the Dermis layer.

### Dosage

A topical composition, in accordance with embodiments of the present invention, may for example be applied by hand to the skin of a subject by spreading a layer (e.g. very thin layer) of the composition (e.g. in a cream form), optionally in circular movements. The topical composition may, for example, be administered/applied once a day before going to sleep.

### EXAMPLES

The following is an illustrative, non-limiting, example of a combination of substances included in a topical composition, in accordance with some embodiments—including optional concentration level ranges for each of the included active substances.

Active and functional substances/materials: (1) Shea Butter—about 1% to about 4% by weight; (2) Persea gratissima—about 1% to about 4% by weight; (3) Pink Pepperlslim—about 0.01% to about 3% by weight; (4) Hamamelis—about 0.5% to about 4% by weight; (5) Aloe Vera—about 1% to about 5% by weight; (6) Wild Yam—about 0.1% to about 2% by weight; (7) Oat—about 0.15% to about 3% by weight; (8) Black Kohosh—about 0.2% to about 3.5% by weight; and/or (9) Nettle—about 0.05% to about 2% by weight.

Base substances/materials of the composition: (1) Alcohol—Isopropyl, Propanol, Propylene Glycol and/or other; (2) Acids—Acetic acid, Alpha acid and/or other; (3) Preservatives—Organic acid, Phenoxyethanol, Sothiazolinones, Formaldehyde, Parabens, Ethylhexylglycerin, BHT (Butylated Hydroxytoluene) and/or other; and/or (4) Vitamins—Vitamin E, Vitamin C and/or other.

The following is an illustrative, non-limiting, example of a specific topical biocompatible composition, in accordance with some embodiments of the present invention.

Table 1, shown below, is an example of a topical composition, in accordance with some embodiments of the present invention, wherein material/substances collectively comprising the composition, their respective relative amounts within the entire composition, their categories and their function as part of the composition, have been listed.

TABLE 1

| | | Optional | | | |
|---|---|---|---|---|---|
| | Material | Supplier | Category | % | Function |
| 1 | Aqua | In-House(IH) | Water | 60 | |
| 2 | Alcohol | DEA | Cetyl Solvent | 4 | Mixing oil and water |
| 3 | Shea | Herb | Butter | 2.5 | Skin relaxing/Thickener |
| 4 | Lavender | IH | Oil | 1 | Skin Con. |
| 5 | Aloe Vera | IH | Jelly Extract | 1.2 | Skin Soothing |
| 6 | Glycerol | GCM | Oil | 1.2 | Desiccant |
| 7 | Acrylates | IH | Acid | 1 | Fixative |
| 8 | Persea Gratissima | DEA | Oil | 1 | Cream |
| 9 | Sodium Hyd. | ASS | Hydroxide | 0.8 | Base |
| 10 | Hamamelis | Venda | Cream | 2 | Skin Stimulation |
| 11 | Pink pepperslim | IH | Extract | 1.6 | Dermis act. |
| 12 | Wild Yam | SHEY | Extract | 1 | Blood Flow |
| 13 | Nettle | IH | Extract | 1 | cooper's reviling |
| 14 | Oat | Holon | Extract | 2 | Woman period effect |
| 15 | Black Kohosh | IH | Extract | 2 | Hormonally treatment |
| 16 | Phenoxy | IH | Thanol | 2 | preservative |
| 17 | Ethiylhexyl | IH | glycerin | 2 | Aither Preservative |
| 18 | Lecitine | IH | cream/oil | 2 | Pigments Merge |
| 19 | Xasanthan gum | IH | Past | 2.5 | Mastic separation block |
| 20 | Butyphenyl | Mugler/IH | Extract | 1.4 | Fragrance |
| 21 | Methylpropional | Mugler/IH | Extract | 1.4 | Fragrance |
| 22 | Coumarin | Mugler/IH | Extract | 1.4 | Fragrance |
| 23 | Benzyl salicylate | Mugler/IH | Extract | 1 | Fragrance |
| 24 | Linalool | Mugler/IH | Extract | 1 | Fragrance |
| 25 | Geraniol | Mugler/IH | Extract | 1 | Fragrance |
| 26 | Hexyl cinnamal | Mugler/IH | Extract | 1 | Fragrance |
| 27 | Citronellol | Mugler/IH | Extract | 1 | Fragrance |
| | | | | 100% | |

According to some embodiments of the present invention, a topical composition may comprise a combination of active materials including, or derived from: Pink Pepperslim (Schinus terebinthifolius) being present in the composition at an active material amount of from about 0.1% to about 2.5% by weight; Black Cohosh (Actaea racemosa) being present in the composition at an active material amount of from about 0.1% to about 2% by weight; and Nettle (Urtica dioica) being present in the composition at an active material amount of from about 0.1% to about 2% by weight.

According to some embodiments, the topical composition may further comprise one or more active materials selected from the group consisting of: Shea Butter, Persea gratissima, Hamamelis (Witch-hazel), Aloe Vera, Wild Yam (Dioscorea villosa) and Oat (Avena sativa).

According to some embodiments, Shea Butter may be present in the composition at an amount of from about 2% to about 3% by weight. According to some embodiments, Persea gratissima may be present in the composition at an amount of from about 0.5% to about 1.5% by weight. According to some embodiments, Hamamelis (Witch-hazel) may be present in the composition at an amount of from about 1.5% to about 2.5% by weight. According to some embodiments, Aloe Vera may be present in the composition at an amount of from about 1% to about 2% by weight. According to some embodiments, Wild Yam (Dioscorea villosa) may be present in the composition at an amount of from about 0.5% to about 1.5% by weight. According to some embodiments, Oat (Avena sativa) may be present in the composition at an amount of from about 1.5% to about 2.5% by weight.

According to some embodiments, the topical composition may further comprise the following base materials: alcohol, selected from the group consisting of: Isopropyl, Propanol and/or Propylene Glycol; acid, selected from the group consisting of: Acetic acid and/or Alpha acid; preservative, selected from the group consisting of: organic acid, Phenoxyethanol, Sothiazolinones, Formaldehyde, Parabens, Ethylhexylglycerin, and/or BHT (Butylated Hydroxytoluene); and/or vitamin, selected from the group consisting of: vitamin E and/or vitamin C.

According to some embodiments, the topical composition may further comprise one or more agents selected from the group consisting of: a carrier, a solvent, a chelating agent, a humectant, a thickener, an emollient, an emulsifier, a skin conditioning agent, an antioxidant, an anti-inflammatory agent, a pH adjuster and/or a preservative.

According to some embodiments, the topical composition may further comprise one or more agents selected from the group consisting of: an excipient, a filler and/or a diluent.

According to some embodiments, the topical composition may further comprise deionized water or pasteurized water, wherein the water may be present in the composition at an amount of from about 65% to about 75% by weight.

According to some embodiments, the topical composition may further comprise an insect repellent, wherein the relative amount of insect repellent within the topical composition is set reverse-proportionally to the amount of Black Cohosh (Actaea racemosa) it includes.

According to some embodiments, the topical composition may further comprise one or more fragrances selected from the group consisting of: Butylphenyl, Methylpropional, Coumarin, Benzyl Salicylate, Limonene, Linalool, Geraniol, Amyl Cinamal, Citronellol and/or Hexy Cinamal.

According to some embodiments, the topical composition may further comprise one or more pigments selected from the group consisting of: brown pigment, caramel pigment, mocha pigment and/or bez pigment, wherein the selected one or more pigments collectively give the topical composition a skin like color or shade.

According to some embodiments of the present invention, a method for treating skin wrinkling, may comprise: topically administering to skin area of a subject in need thereof a therapeutically effective amount of a topical composition comprising a combination of active materials including, or derived from: Pink Pepperslim (Schinus terebinthifolius) being present in the composition at an active material amount of from about 0.1% to about 2.5% by weight; Black Cohosh (Actaea racemosa) being present in the composition at an active material amount of from about 0.1% to about 2% by weight; and Nettle (Urtica dioica) being present in the composition at an active material amount of from about 0.1% to about 2% by weight;

According to some embodiments, the method administered topical composition may be in the form of a cream or a lotion. According to some embodiments, administering may comprise one daily application of the topical composition. According to some embodiments, administering one daily application of the topical composition may be performed just prior to the subject's sleep time. According to some embodiments, the method may further comprise cleaning the skin prior to administration of the topical composition.

The subject matter described above is provided by way of illustration only and should not be constructed as limiting. While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. A topical composition comprising a combination of active materials including, or derived from:
Pink Pepperslim (Schinus terebinthifolius) being present in said composition at an active material amount of from about 0.1% to about 2.5% by weight;
Black Cohosh (Actaea racemosa) being present in said composition at an active material amount of from about 0.1% to about 2% by weight; and
Nettle (Urtica dioica) being present in said composition at an active material amount of from about 0.1% to about 2% by weight.

2. The topical composition according to claim 1, further comprising one or more active materials selected from the group consisting of: Shea Butter, Persea gratissima, Hamamelis (Witch-hazel), Aloe Vera, Wild Yam (Dioscorea villosa) and Oat (Avena sativa).

3. The topical composition according to claim 2, wherein Shea Butter is being present in said composition at an amount of from about 2% to about 3% by weight.

4. The topical composition according to claim 2, wherein Persea gratissima is being present in said composition at an amount of from about 0.5% to about 1.5% by weight.

5. The topical composition according to claim 2, wherein Hamamelis (Witch-hazel) is being present in said composition at an amount of from about 1.5% to about 2.5% by weight.

6. The topical composition according to claim 2, wherein Aloe Vera is being present in said composition at an amount of from about 1% to about 2% by weight.

7. The topical composition according to claim 2, wherein Wild Yam (Dioscorea villosa) is being present in said composition at an amount of from about 0.5% to about 1.5% by weight.

8. The topical composition according to claim 2, wherein Oat (Avena sativa) is being present in said composition at an amount of from about 1.5% to about 2.5% by weight.

9. The topical composition according to claim 1, further comprising the following base materials: alcohol, selected from the group consisting of: Isopropyl, Propanol and Propylene Glycol; acid, selected from the group consisting of: Acetic acid and Alpha acid; preservative, selected from the group consisting of: organic acid, Phenoxyethanol, Sothiazolinones, Formaldehyde, Parabens, Ethylhexylglycerin, and BHT (Butylated Hydroxytoluene); and vitamin, selected from the group consisting of: vitamin E and vitamin C.

10. The topical composition according to claim 1, further comprising one or more agents selected from the group consisting of: a carrier, a solvent, a chelating agent, a humectant, a thickener, an emollient, an emulsifier, a skin conditioning agent, an antioxidant, an anti-inflammatory agent, a pH adjuster and a preservative.

11. The topical composition according to claim 10, further comprising one or more agents selected from the group consisting of: an excipient, a filler and a diluent.

12. The topical composition according to claim 1, further comprising deionized water or pasteurized water, wherein said water is being present in said composition at an amount of from about 65% to about 75% by weight.

13. The topical composition according to claim 1, further comprising an insect repellent, wherein the relative amount of insect repellent within said topical composition is set reverse-proportionally to the amount of Black Cohosh (Actaea racemosa) it includes.

14. The topical composition according to claim 2, further comprising one or more fragrances selected from the group consisting of: Butylphenyl, Methylpropional, Coumarin, Benzyl Salicylate, Limonene, Linalool, Geraniol, Amyl Cinamal, Citronellol and Hexy Cinamal.

15. The topical composition according to claim 2, further comprising one or more pigments selected from the group consisting of: brown pigment, caramel pigment, mocha pigment and bez pigment, wherein the selected one or more pigments collectively give said topical composition a skin like color or shade.

16. A method for treating skin wrinkling, said method comprising:
topically administering a therapeutically effective amount of a topical composition comprising a combination of active materials including, or derived from:
(a) Pink Pepperslim (Schinus terebinthifolius) being present in said composition at an active material amount of from about 0.1% to about 2.5% by weight;
(b) Black Cohosh (Actaea racemosa) being present in said composition at an active material amount of from about 0.1% to about 2% by weight; and
(c) Nettle (Urtica dioica) being present in said composition at an active material amount of from about 0.1% to about 2% by weight;
to skin area of a subject in need thereof.

17. The method of claim 16, wherein the topical composition is in the form a cream or a lotion.

18. The method of claim 17, wherein administering comprises one daily application of the topical composition.

19. The method of claim 18, wherein administering one daily application of the topical composition is performed in just prior to the subject's sleep.

20. The method of claim 19, further comprising cleaning the skin prior to administration of the topical composition.
